(19) United States
(12) Patent Application Publication
(10) Pub. No.: US 2007/0298486 A1
(43) Pub. Date: Dec. 27, 2007

(54) Microchannel Apparatus and Methods Of Conducting Unit Operations With Disrupted Flow

(75) Inventors: Ravi Arora, New Albany, OH (US); Anna Lee Tonkovich, Dublin, OH (US); Dongming Qiu, Dublin, OH (US); Laura J. Silva, Dublin, OH (US)

Correspondence Address: FRANK ROSENBERG, P.O. BOX 29230, SAN FRANCISCO, CA, 94129-0230, US

(73) Assignee: Velocys Inc., Plain City, OH (US)

(21) Appl. No.: 11/763,336

(22) Filed: Jun. 14, 2007

Related U.S. Application Data
(60) Provisional application No. 60/805,072, filed on Jun. 16, 2006.

(51) Int. Cl. C12M 3/00

(52) U.S. Cl. 435/287.1

(57) ABSTRACT

The invention described herein concerns microchannel apparatus that contains, within the same device, at least one manifold and multiple connecting microchannels that connect with the manifold. For superior heat or mass flux in the device, the volume of the connecting microchannels should exceed the volume of manifold or manifolds. Methods of conducting unit operations in microchannel devices having simultaneous disrupted and non-disrupted flow through microchannels is also described.

Conducting chemical processes in microchannels is well known to be advantageous for enhanced heat and mass transfer. Many researchers have shown that the heat and the mass transfer in microchannels are enhanced as the dimensions are made smaller. Nishio (2003) published that the work at Institute of Industrial Science, the University of Tokyo had shown that the results for microchannel tubes larger than 0.1 mm in inner diameter are in good agreement with the conventional analyses. The article also presents the heat transfer coefficient as a function of tube diameter using conventional correlations and shows that as the diameter of tube decreases, the heat transfer coefficient increases. Thus, the prior art teaches that smaller tube diameters give better heat transfer performance.

Guo et al. (2003) published an article on size effect on single phase flow and heat transfer at microscale. One of the conclusions of the study was “Discrepancy between experimental results for the friction factor and the Nusselt number and their standard value (conventional value) due to the measurement errors or entrance effects might be misunderstood as being caused by novel phenomenon at micro scale”. He also pointed out that the smaller diameter channel results in large surface area to volume ratio which provides higher Nusselt number as well as friction factor.

It is generally accepted that microchannels are conventionally designed for operation in the laminar flow regime. Pan et al. (2007) have stated in an article accepted (published online) by Chemical Engineering Journal “In practice, flow velocities in microchannels are usually lower than 10 m/s and the hydraulic diameters are no more than 500 μm, so the Reynolds number is lower than 2000”. It has also been proven by several researchers (Hrnjak etal (2006)) that the critical Reynolds number for flow regime transition from laminar to transition flow regime in microchannel with critical dimension greater than 0.05 mm follows conventional values which is ˜2000.

Vogel in 2006 published a heat exchanger design method. Heat enhancement was obtained by keeping the flow in the developing regime which provides high heat transfer coefficient. The method teaches to keep the L/D ratio under 100 for better heat transfer performance. However this approach would result in short connecting channel length; hence small connecting channel pressure drop. For a scale-up device, the approach may require large number of channels and corresponding large manifolds.

Delsman etal in 2004 studied the effect of the manifold geometry and the total flow rate on flow distribution through Computational Fluid Dynamics models. The dimensions of the connecting channel (cross-section) were fixed (0.4 mm×0.3 mm). The total number of channels in the analysis was 19. The analysis focused on modifying the shape of the manifold to obtain a uniform flow distribution. The analysis showed clearly that the mal-distribution increases as the velocity through the manifold increases. Applying this approach to a scale up design, where the total number of connecting channels is large (≧100) and the flow rate would be large will result in large manifold volume.

Tonomura etal in 2004 also studied the optimization of microdevices using Computational Fluid Dynamics models. The total number of channels in the analysis was 5. The study showed that the shaped manifolds improve the flow distribution for given connecting channel dimensions but the manifold and connecting channels were not designed together for the application. The optimization in the study was based on reducing the overall manifold flow area rather than the whole device. With this approach, a scale-up unit (with a large (≧15 cm) manifold length, or a large number of connecting channels) will again end up with large manifold dimensions as the connecting channel design is not included in the optimization.

Amador etal in 2004 used the electrical resistance network approach to analyze flow distribution in different microreactor scale-out geometries. The article presented a system of equation for analyzing consecutive and bifurcation manifold structures. The presented system of equations for analysis is applicable for the laminar regime only. The article presented a method to calculate the required dimensional ratios to achieve given flow distribution for laminar regime in the manifold as well as connecting channels.

Webb in 2003 studied the effect of manifold design on flow distribution in parallel microchannels. The article demonstrated an approach of designing the manifold flow area greater or equal to the sum of flow area of all connecting channels to obtain uniform flow distribution. Applying this approach to scaled up microchannel units will result in large manifolds as the number of connecting channel increases.

Chong et al. in 2002 published a modeling approach by employing thermal resistance network for optimizing the microchannel heat sink design. The results showed that the heat sink design operating in the laminar regime outperforms the heat sink design in turbulent regime. The article does not discuss the implication of design on manifold size.

### SUMMARY OF THE INVENTION

In the prior art, the connecting microchannel dimensions may be set based on the heat transfer or mass transfer requirements. For example, for a heat exchanger unit design, the connecting channel dimensions may be determined based on the overall heat transfer requirements. Generally, a smaller gap for laminar flow gives better heat transfer coefficient and compact connecting channel size, the smallest dimensions of connecting channels are on the order of 2 mm or less, and more preferably less than 0.25 mm preferred to maximize heat transfer. Afterwards the manifold may be designed to obtain a uniform flow distribution in multiple channels while meeting the overall pressure drop constraint. Generally the smallest dimension or manifold gap available for the manifold section is similar in dimension to the smallest dimension of the connecting channels. The advantage of microchannel architecture lies in the small dimensions, generally the drive is to keep the smallest dimension as small as possible in the connecting channels.

With the smaller channel gaps, the velocity in the manifold section is high leading to large momentum effects, manifold pressure drop and flow mal-distribution. The common approach to reduce the mal-distribution and pressure drop is to increase the open flow area in the manifold which increases the width and therefore the size of the manifold section. Applying this approach to a commercial unit will result in a large manifold section compared to connecting microchannel section.

In the present invention, microchannel apparatus is designed with control of both connecting channels and manifolds for heat and/or mass transfer with disrupted flow in at least a portion of the connecting channels.

In a first aspect, the invention provides a method of conducting a unit operation in an integrated microchannel apparatus, comprising: passing a fluid in an apparatus; wherein the apparatus comprises a manifold connected to plural connecting microchannels; wherein the manifold's volume is less than the volume of the plural connecting microchannels; and wherein the manifold's length is at least 15 cm or wherein there are at least 100 connecting channels connected to the manifold; controlling conditions such that the fluid is in disrupted flow through at least a portion of the connecting microchannels; and conducting a unit operation on the fluid in the connecting microchannels. Disrupted flow occurs for at least a portion of the length of one or more of the connecting channels, preferably this portion comprises at least 5% of the connecting channel length, more preferably at least 20%, more preferably at least 50%, and in some embodiments at least 90% of the connecting channel length; and, preferably, the plural connecting channels comprise at least 10, more preferably at least 20, and in some embodiments at least 100 connecting channels, in which each connecting channel has disrupted flow occurring in at least 5% (or at least 20%, or at least 50%, or at least 90%) of it's length (and in some embodiments there is disrupted flow in all of the plural connecting channels).

In some embodiments, the manifold is a header and the header has an inlet, and fluid passes through the header inlet at a Reynold's number greater than 2200 (or at least 2000 or at least 2200). In some embodiments, flow through the connecting channels has a Reynolds number of at least 2200. In some embodiments, the integrated microchannel apparatus (and/or the method) of the present invention has a heat duty greater than 0.01 MW. In some embodiments, pressure drop through the manifold is less than or equal to the average pressure drop through the plural connecting channels. In some embodiments, the manifold is a header and wherein the pressure drop in the manifold, that is between the header inlet and the connecting channel inlet (corresponding to a header outlet) having the lowest pressure, is less than 50% (or less than 25%) of the pressure drop through the plural connecting channels (measured as an average pressure drop). In some embodiments, the manifold volume is less than 50% (or less than 25%) of the volume of the plural connecting channels. In some embodiments, the integrated microchannel apparatus has a heat duty greater than 0.1 MW, more preferably at least 1 MW. In preferred embodiments, there are no orifices controlling flow between the manifold and the connecting channels. An orifice's cross-sectional area is less than 20%, or preferably less than 10% of the average cross-sectional area of the connecting channels.

In some embodiments, the manifold includes at least two sections. In some embodiments, the manifold includes a first section that is an open manifold and the second section that includes a submanifold, gate, or grate.

In some preferred embodiments, flow through the plural connecting channels is in transitional or turbulent flow. In some preferred embodiments, the plural connecting channels have smooth walls and preferably do not have surface features or other obstructions; and in some embodiments, do not include a catalyst. In some preferred embodiments, the manifold comprises a manifold inlet and comprising a flow path through the manifold inlet and through the plural connecting channels; and the flow path does not include any orifices, gates, grates, or flow straighteners.

Any of the embodiments of the invention can be more specifically described as consisting essentially of, or consisting of a set of components or steps. For example, in a preferred embodiment, the invention comprises a manifold inlet and a flow path through the manifold inlet and through the plural connecting channels wherein the flow path consists essentially of manifolds, submanifolds, and connecting channels.

In some preferred embodiments, there are at least 200 connecting microchannels connected to the manifold. In some preferred embodiments, the connecting microchannels have a minimum dimension (typically a gap in a laminated device) in the range of 0.5 to 1.5 mm, in some embodiments in the range of 0.7 to 1.2 mm. In some preferred embodiments, the manifold has a minimum dimension in the range of 0.5 to 1.5 mm; typically this is within the thickness of a single sheet in a laminated device.

In some preferred embodiments, the plural connecting microchannels comprise a solid catalyst.

In some embodiments, there is turbulent flow in at least 90% of the connecting channels, in some embodiments there is turbulent flow in all of the plural connecting channels.

In a related aspect, the device comprises at least two manifolds, a first manifold and a second manifold, wherein the first manifold is connected to a first set of plural connecting microchannels and the second manifold is connected to a second set of plural connecting microchannels. In this method, a first fluid can flow through the first manifold and in disrupted flow (at least partly, preferably substantially) through the first set of connecting microchannels and a second fluid flows through the second manifold and flows in non-disrupted flow (at least partly, preferably substantially) through the second set of connecting microchannels. The first and second fluids can be of the same type or of different types. In this case, unlike the first aspect, the manifold can be of any length and can have any number of connecting channels—although in preferred embodiments it has a length greater than 15 cm and/or at least 100 connecting channels.

In another aspect the invention provides a method of conducting a unit operation in an integrated microchannel apparatus, comprising: passing a fluid in an apparatus; wherein the apparatus comprises a manifold connected to plural connecting microchannels; wherein the manifold's volume is less than the volume of the plural connecting microchannels; controlling conditions such that the fluid is in disrupted flow (at least partly, preferably substantially) through at least some the plural connecting microchannels and controlling conditions such that the fluid is in non-disrupted flow (at least partly, preferably substantially) through at least some other of the plural connecting microchannels; and conducting a unit operation on the fluid in the connecting microchannels (both in the disrupted and non-disrupted flows). For example, a manifold could have at least 10 connecting channels with 6 or more connecting channels in disrupted flow and 4 or more in non-disrupted flow, such as by using surface features or obstacles in some of the connecting channels and smooth walls in some other of the connecting channels.

In another aspect, the invention provides microchannel apparatus, comprising: a manifold connected to plural connecting microchannels; wherein the manifold's volume is less than the volume of the plural connecting microchannels; and wherein the manifold's length is at least 15 cm or wherein there are at least 100 connecting channels connected to the manifold. In a preferred embodiment, the apparatus includes at least 10 layers of heat exchange microchannel arrays interfaced with at least 10 layers of reaction microchannels. In some embodiments, the reaction microchannels comprise a catalyst wall coating. In preferred embodiments, each layer of heat exchange microchannel arrays comprises a manifold and an array of heat exchange connecting microchannels connected to the manifold. Preferably the manifold in each layer is substantially limited to that layer and does not extend over plural layers of heat exchange microchannel arrays and/or reaction microchannel arrays. In some embodiments, a manifold extends over plural layers of heat exchange microchannel arrays such that plural arrays of heat exchange connecting microchannels in plural layers connect to the manifold.

In another aspect, the invention provides a microchannel system comprising a device and a fluid, comprising: a manifold connected to plural connecting microchannels; wherein the manifold's volume is less than the volume of the plural connecting microchannels; wherein the manifold's length is at least 15 cm or wherein there are at least 100 connecting channels connected to the manifold; and the system also comprises a fluid passing through the connecting microchannels in disrupted flow for at least a portion of the length. This system may have any of the characteristics mentioned herein for any of the inventive methods.

In various embodiments, the invention provides higher heat flux or higher mass transfer.

### GLOSSARY

Structural features related to manifolding are as defined in U.S. Published Patent Application No. 20050087767, filed Oct. 27, 2003 and U.S. patent application Ser. No. 11/400,056, filed Apr. 11, 2006. Surface features and general device construction are as defined in U.S. patent application Ser. No. 11/388,792, filed Mar. 23, 2006. All of these patent applications are incorporated herein by reference as if reproduced in full below. In cases where the definitions set forth here are in conflict with definitions in the patent applications referred to above, then the definitions set forth here are controlling.
As is standard patent terminology, “comprising” means “including” and neither of these terms exclude the presence of additional or plural components. For example, where a device comprises a lamina, a sheet, etc., it should be understood that the inventive device may include multiple laminae, sheets, etc. In alternative embodiments, the term “comprising” can be replaced by the more restrictive phrases “consisting essentially of” or “consisting of.”
Channels are defined by channel walls that may be continuous or may contain gaps.
Interconnecting pathways through a monolith foam or felt are not connecting channels (although a foam, etc. may be disposed within a channel).
“Connecting channels” are channels connected to a manifold. Typically, unit operations occur in connecting channels. Connecting channels have an entrance cross-sectional plane and an exit cross-sectional plane. Although some unit operations or portions of unit operations may occur in a manifold, in preferred embodiments, greater than 70% (in some embodiments at least 95%) of a unit operation occurs in connecting channels. A “connecting channel matrix” is a group of adjacent, substantially parallel connecting channels. In preferred embodiments, the connecting channel walls are straight. The connecting channel pressure drop is the static pressure difference between the center of the entrance cross-sectional plane and the center of the exit cross-sectional plane of the connecting channels, averaged over all connecting channels. In some preferred embodiments, connecting channels are straight with substantially no variation in direction or width. The connecting channel pressure drop for a system of multiple connecting channels is the arithmetic mean of each individual connecting channel pressure drop. That is, the sum of the pressure drops through each channel divided by the number of channels. “Connecting microchannels” have a minimum dimension of 2 mm or less, more preferably 0.5 to 1.5 mm, still more preferably 0.7 to 1.2 mm, and a length of at least 1 cm.
“Disrupted flow” means transitional or turbulent flow in smooth microchannels and also includes flow through a microchannel having surface features. Disrupted flow occurs for at least a portion of the length of a connecting channel, preferably at least 5% of the connecting channel length, more preferably at least 20%, more preferably at least 50%, and in some embodiments at least 90% of the connecting channel length. Surface features are described in U.S. patent application Ser. No. 11/388,792 and typically contain chevrons or other shapes recessed into a channel wall that aid in fluid mixing so that good mixing occurs without the high Reynold's numbers of turbulent or transitional flow. Surface features may also be used for Reynolds numbers greater than 2200 or for transition or turbulent flow. Disrupted flow may also be created by obstructions or projections or recesses in the main channel that force the fluid motion to deviate from a typical laminar or straight flow profile. Disrupted flow may also be created by three dimensionally tortuous flow paths in a connecting channel that create flow rotation, secondary vortices or other angled or orthogonal flow vectors relative to the main direction of flow. The flow deviations or non-straight flow paths are particularly advantageous for enhancing heat transfer to the wall, mass transfer to the wall, or chemical reaction at either the wall or homogeneously in the fluid phase.
“Disrupted flow substantially through the connecting channels” means that flow is substantially disrupted for the length in the region of a microchannel where a unit operation occurs (preferably at least 90% of the length in the region of a microchannel where a unit operation occurs). Disrupted flow is not merely caused by exit or entrance effects (i.e. the length over which the velocity distribution changes and hydrodynamic boundary layer develops).

A “gate” comprises an interface between the manifold and two or more connecting channels. A gate has a nonzero volume. A gate controls flow into multiple connecting channels by varying the cross sectional area of the entrance to the connecting channels. A gate is distinct from a simple orifice, in that the fluid flowing through a gate has positive momentum in both the direction of the flow in the manifold and the direction of flow in the connecting channel as it passes through the gate. In contrast, greater than 75% of the positive momentum vector of flow through an orifice is in the direction of the orifice's axis. A typical ratio of the cross sectional area of flow through a gate ranges between 2-98% (and in some embodiments 5% to 52%) of the cross sectional area of the connecting channels controlled by the gate including the cross sectional area of the walls between the connecting channels controlled by the gate. The use of two or more gates allows use of the manifold interface's cross sectional area as a means of tailoring manifold turning losses, which in turn enables equal flow rates between the gates. These gate turning losses can be used to compensate for the changes in the manifold pressure profiles caused by friction pressure losses and momentum compensation, both of which have an effect upon the manifold pressure profile. The maximum variation in the cross-sectional area divided by the minimum area, given by the Ra number, is preferably less than 8, more preferably less than 6 and in even more preferred embodiments less than 4.

A “grate” is a connection between a manifold and a single channel. A grate has a nonzero connection volume. In a shim construction a grate is formed when a cross bar in a first shim is not aligned with a cross bar in an adjacent second shim such that flow passes over the cross bar in the first shim and under the cross bar in the second shim.
“Heat duty” is defined by the total heat measured in Watts that is transferred in a device and is preferentially greater than 10 kW and preferably ranges from 10 kW to 100 MW in an integrated microchannel unit apparatus.
A “header” is a manifold arranged to deliver fluid to connecting channels.
A “height” is a direction perpendicular to length. In a laminated device, height is the stacking direction.
A “hydraulic diameter” of a channel is defined as four times the cross-sectional area of the channel divided by the length of the channel's wetted perimeter.
An “L-manifold” describes a manifold design where flow direction into one manifold is normal to axes of the connecting channel, while the flow direction in the opposite manifold is parallel with the axes of the connecting channels: For example, a header L-manifold has a manifold flow normal to the axes of the connecting channels, while the footer manifold flow travels in the direction of connecting channels axes out of the device. The flow makes an “L” turn from the manifold inlet, through the connecting channels, and out of the device. When two L-manifolds are brought together to serve a connecting channel matrix, where the header has inlets on both ends of the manifold or a footer has exits from both ends of the manifold, the manifold is called a “T-manifold”.
A “laminated device” is a device made from laminae that is capable of performing a unit operation on a process stream that flows through the device.
A “length” refers to the distance in the direction of a channel's (or manifold's) axis, which is in the direction of flow.
“M2M manifold” is defined as a macro-to-micro manifold, that is, a microchannel manifold that distributes flow to or from one or more connecting microchannels. The M2M manifold in turn takes flow to or from another larger cross-sectional area delivery source, also known as macro manifold. The macro manifold can be, for example, a pipe, a duct or an open reservoir.
A “manifold” is a volume that distributes flow to two or more connecting channels. The entrance, or inlet, surface of a header manifold is defined as the surface in which marks a significant difference in header manifold geometry from the upstream channel. The exit, or outlet, surface of the footer manifold is defined as the surface which marks a significant difference in the footer manifold channel from the downstream channel. For rectangular channels and most other typical manifold geometries, the surface will be a plane; however, in some special cases such as hemicircles at the interface between the manifold and connecting channels it will be a curved surface. A significant difference in manifold geometry will be accompanied by a significant difference in flow direction and/or mass flux rate. A manifold includes submanifolds if the submanifolding does not cause significant difference in flow direction and/or mass flux rate. A microchannel header manifold's entrance plane is the plane where the microchannel header interfaces a larger delivery header manifold, such as a pipe or duct, attached to a microchannel device through welding a flange or other joining methods. In most cases, a person skilled in this art will readily recognize the boundaries of a manifold that serves a group of connecting channels.

Manifolds can be L, U or Z types. In a “U-manifold,” fluid in a header and footer flow in opposite directions while being at a non zero angle to the axes of the connecting channels.

For a header the “manifold pressure drop” is the static pressure difference between the arithmetic mean of the area-averaged center pressures of the header manifold inlet planes (in the case where there is only one header inlet, there is only one inlet plane) and the arithmetic mean of each of the connecting channels' entrance plane center pressures. The header manifold pressure drop is based on the header manifold entrance planes that comprise 95% of the net flow through the connecting channels, the header manifold inlet planes having the lowest flow are not counted in the arithmetic mean if the flow through those header manifold inlet planes is not needed to account for 95% of the net flow through the connecting channels. The header (or footer) manifold pressure drop is also based only on the connecting channels' entrance (or exit) plane center pressures that comprise 95% of the net flow through the connecting channels, the connecting channels' entrance (or exit) planes having the lowest flow are not counted in the arithmetic mean if the flow through those connecting channels is not needed to account for 95% of the net flow through the connecting channels. For a footer, the manifold pressure drop is the static pressure difference between the arithmetic mean of each of the connecting channel's exit plane center pressures and the arithmetic mean of the area-averaged center pressures of the footer manifold outlet planes (in the case where there is only one header outlet, there is only one outlet plane). The footer manifold pressure drop is based on the footer manifold exit planes that comprise 95% of the net flow through the connecting channels, the footer manifold outlet planes with the lowest flow are not counted in the arithmetic mean if the flow through those exit planes is not needed to account for 95% of the net flow through the connecting channels. If a manifold has more than one sub-manifold, the manifold pressure drop is based upon the number average of sub-manifold values.

A “microchannel” is a channel having at least one internal dimension (wall-to-wall, not counting catalyst) of 10 mm or less (preferably 2.0 mm or less) and greater than 1 μm (preferably greater than 10 μm), and in some embodiments 50 to 500 μm. Microchannels are also defined by the presence of at least one inlet that is distinct from at least one outlet. Microchannels are not merely channels through zeolites or mesoporous materials. The length of a microchannel corresponds to the direction of flow through the microchannel. Microchannel height and width are substantially perpendicular to the direction of flow of through the channel. In the case of a laminated device where a microchannel has two major surfaces (for example, surfaces formed by stacked and bonded sheets), the height is the distance from major surface to major surface and width is perpendicular to height.

The value of the Reynolds number describes the flow regime of the stream. While the dependence of the regime on Reynolds number is a function of channel cross-section shape and size, the following ranges are typically used for channels:
Laminar: Re<2000 to 2200
Transition: 2000-2200<Re<4000 to 5000
Turbulent: Re>4000 to 5000.

A “subchannel” is a channel that is within a larger channel. Channels and subchannels are defined along their length by channel walls.

A “sub-manifold” is a manifold that operates in conjunction with at least one other submanifold to make one large manifold in a plane. Sub-manifolds are separated from each other by continuous walls.

A “surface feature” is a projection from, or a recess into, a microchannel wall that modify flow within the microchannel. If the area at the top of the features is the same or exceeds the area at the base of the feature, then the feature may be considered recessed. If the area at the base of the feature exceeds the area at the top of the feature, then it may be considered protruded (except for CRFs discussed below). The surface features have a depth, a width, and a length for non-circular surface features. Surface features may include circles, oblong shapes, squares, rectangles, checks, chevrons, zig-zags, and the like, recessed into the wall of a main channel. The features increase surface area and create convective flow that brings fluids to a microchannel wall through advection rather than diffusion. Flow patterns may swirl, rotate, tumble and have other regular, irregular and or chaotic patterns—although the flow pattern is not required to be chaotic and in some cases may appear quite regular. The flow patterns are stable with time, although they may also undergo secondary transient rotations. The surface features are preferably at oblique angles—neither parallel nor perpendicular to the direction of net flow past a surface. Surface features may be orthogonal, that is at a 90 degree angle, to the direction of flow, but are preferably angled. The active surface features are further preferably defined by more than one angle along the width of the microchannel at least at one axial location. The two or more sides of the surface features may be physically connected or disconnected. The one or more angles along the width of the microchannel act to preferentially push and pull the fluid out of the straight laminar streamlines. Preferred ranges for surface feature depth are less than 2 mm, more preferrably less than 1 mm, and in some embodiments from 0.01 mm to 0.5 mm. A preferred range for the lateral width of the surface feature is sufficient to nearly span the microchannel width (as shown in the herringbone designs), but in some embodiments (such as the fill features) can span 60% or less, and in some embodiments 40% or less, and in some embodiments, about 10% to about 50% of the microchannel width. In preferred embodiments, at least one angle of the surface feature pattern is oriented at an angle of 10°, preferably 30°, or more with respect to microchannel width (90° is parallel to length direction and 0° is parallel to width direction). Lateral width is measured in the same direction as microchannel width. The lateral width of the surface feature is preferably 0.05 mm to 100 cm, in some embodiments in the range of 0.5 mm to 5 cm, and in some embodiments 1 to 2 cm.

“Unit operation” means chemical reaction, vaporization, compression, chemical separation, distillation, condensation, mixing, heating, or cooling. A “unit operation” does not mean merely fluid transport, although transport frequently occurs along with unit operations. In some preferred embodiments, a unit operation is not merely mixing.

The volume of a connecting channel or manifold is based on open space. The volume includes depressions of surface features. The volume of gate or grate features (which help equalize flow distribution as described in the incorporated published patent application) are included in the volume of manifold; this is an exception to the rule that the dividing line between the manifold and the connecting channels is marked by a significant change in direction. Channel walls are not included in the volume calculation. Similarly, the volume of orifices (which is typically negligible) and flow straighteners (if present) are included in the volume of manifold.

In a “Z-manifold,” fluid in a header and footer flow in the same direction while being at a non zero angle to the axes of the connecting channels. Fluid entering the manifold system exits from the opposite side of the device from where it enters. The flow essentially makes a “Z” direction from inlet to outlet.

### BRIEF DESCRIPTION OF THE DRAWINGS

FIG. 1 schematically illustrates a manifold, connecting channels and the connections in between on a shim.

FIG. 2 is a cross-sectional view of section A-A of FIG. 1 with (a) partial etching of one side of the shim or (b) partial etching on both sides of the shim.

FIG. 3 shows a sub-manifold with varying cross-section.

FIG. 4 shows rounded corners of the sub-manifolds.

FIG. 5 illustrates a gradual transition from gate to connecting channels.

FIG. 6 shows an alternate connection of connecting channels to exit sub-manifolds.

FIG. 7 illustrates a wall shim.

FIG. 8 shows the assembly of manifold and wall shim to develop a device stack.

FIG. 9 illustrates a wall shim with sub-manifolds.

FIG. 10 illustrates heat exchanger design requirements.

FIG. 11 shows the dimensions of a single repeating unit for small microchannels.

FIG. 12 shows core dimensions for Design 1 for small microchannels.

FIG. 13 shows flow direction in the microchannel unit for Stream A and Stream B in the Examples.

FIG. 14 is a schematic of the strategy used to manifold the designed core.

FIG. 15 is a schematic of a manifold design.

FIG. 16 is a schematic of flow in and out in one of the four core sections in the Examples.

FIG. 17 shows a manifold design for distribution of Stream A flow in one of the four core sections for small microchannels.

FIG. 18 shows dimensions of a single repeating unit for large microchannels

FIG. 19 shows core dimensions for Design 2 with large microchannels.

FIG. 20 illustrates a manifold design for distribution of a stream flowing in one of four core sections for large microchannels.

FIG. 21 shows dimensions of a single repeating unit for large microchannels from the Examples.

FIG. 22 shows the core dimensions for Design 2 with large microchannels.

FIG. 23 shows a design for distributing a stream in one of the four cores.

FIG. 24 shows a graph of overall device volume as a function of channel gap as calculated from the Examples.

### DETAILED DESCRIPTION OF THE INVENTION

Microchannel Apparatus

Microchannel reactors are characterized by the presence of at least one reaction channel having at least one dimension (wall-to-wall, not counting catalyst) of 2 mm or less (in some embodiments about 1.0 mm or less) and greater than 1 μm, and in some embodiments 50 to 500 μm. A catalytic reaction channel is a channel containing a catalyst, where the catalyst may be heterogeneous or homogeneous. A homogeneous catalyst may be co-flowing with the reactants. Microchannel apparatus is similarly characterized, except that a catalyst-containing reaction channel is not required. The gap (or height) of a microchannel is preferably about 2 mm or less, and more preferably 1 mm or less. The length of a reaction channel is typically longer. Preferably, the length is greater than 1 cm, in some embodiments greater than 50 cm, in some embodiments greater than 20 cm, and in some embodiments in the range of 1 to 100 cm. The sides of a microchannel are defined by reaction channel walls. These walls are preferably made of a hard material such as a ceramic, an iron based alloy such as steel, or a Ni—, Co— or Fe-based superalloy such as monel. They also may be made from plastic, glass, or other metal such as copper, aluminum and the like. The choice of material for the walls of the reaction channel may depend on the reaction for which the reactor is intended. In some embodiments, reaction chamber walls are comprised of a stainless steel or Inconel® which is durable and has good thermal conductivity. The alloys should be low in sulfur, and in some embodiments are subjected to a desulfurization treatment prior to formation of an aluminide. Typically, reaction channel walls are formed of the material that provides the primary structural support for the microchannel apparatus. Microchannel apparatus can be made by known methods, and in some preferred embodiments are made by laminating interleaved plates (also known as “shims”), and preferably where shims designed for reaction channels are interleaved with shims designed for heat exchange. Some microchannel apparatus includes at least 10 layers laminated in a device, where each of these layers contain at least 10 channels; the device may contain other layers with less channels.

Microchannel apparatus (such as microchannel reactors) preferably include microchannels (such as a plurality of microchannel reaction channels) and a plurality of adjacent heat exchange microchannels. The plurality of microchannels may contain, for example, 2, 10, 100, 1000 or more channels capable of operating in parallel. In preferred embodiments, the microchannels are arranged in parallel arrays of planar microchannels, for example, at least 3 arrays of planar microchannels. In some preferred embodiments, multiple microchannel inlets are connected to a common header and/or multiple microchannel outlets are connected to a common footer. During operation, heat exchange microchannels (if present) contain flowing heating and/or cooling fluids. Non-limiting examples of this type of known reactor usable in the present invention include those of the microcomponent sheet architecture variety (for example, a laminate with microchannels) exemplified in U.S. Pat. Nos. 6,200,536 and 6,219,973 (both of which are incorporated by reference). Performance advantages in the use of this type of reactor architecture for the purposes of the present invention include their relatively large heat and mass transfer rates, and the substantial absence of any explosive limits. Pressure drops can be low, allowing high throughput and the catalyst can be fixed in a very accessible form within the channels eliminating the need for separation. In some embodiments, a reaction microchannel (or microchannels) contains a bulk flow path. The term “bulk flow path” refers to an open path (contiguous bulk flow region) within the reaction chamber. A contiguous bulk flow region allows rapid fluid flow through the reaction chamber without large pressure drops. Bulk flow regions within each reaction channel preferably have a cross-sectional area of 5×10⁻⁸ to 1×10⁻² m², more preferably 5×10⁻⁷ to 1×10⁻⁴ m². The bulk flow regions preferably comprise at least 5%, more preferably at least 50% and in some embodiments, 30-99% of either 1) the interior volume of a microchannel, or 2) a cross-section of a microchannel.

In many preferred embodiments, the microchannel apparatus contains multiple microchannels, preferably groups of at least 5, more preferably at least 10, parallel channels that are connected in a common manifold that is integral to the device (not a subsequently-attached tube) where the common manifold includes a feature or features that tend to equalize flow through the channels connected to the manifold. Examples of such manifolds are described in U.S. patent application Ser. No. 10/695,400, filed Oct. 27, 2003 which is incorporated herein. In this context, “parallel” does not necessarily mean straight, rather that the channels conform to each other. In some preferred embodiments, a microchannel device includes at least three groups of parallel microchannels wherein the channel within each group is connected to a common manifold (for example, 4 groups of microchannels and 4 manifolds) and preferably where each common manifold includes a feature or features that tend to equalize flow through the channels connected to the manifold.

In devices with multiple manifolds, the invention can be characterized by the volume ratio of one manifold to its connecting microchannels, or characterized by the volumetric sum of plural manifolds and their connecting microchannels. However, if connecting channels are connected to a header and footer, then both the header and footer must be included in the calculation of manifold volume. The volume of the submanifold is included in the volume of the manifold.

Heat exchange fluids may flow through heat transfer microchannels adjacent to process channels (such as reaction microchannels), and can be gases or liquids and may include steam, oil, or any other known heat exchange fluids—the system can be optimized to have a phase change in the heat exchanger. In some preferred embodiments, multiple heat exchange layers are interleaved with multiple reaction microchannels. For example, at least 10 heat exchangers interleaved with at least 10 reaction microchannels and preferably there are 10 layers of heat exchange microchannel arrays interfaced with at least 10 layers of reaction microchannels. Each of these layers may contain simple, straight channels or channels within a layer may have more complex geometries. In preferred embodiments, one or more interior walls of a heat exchange channel, or channels, has surface features.

A general methodology to build commercial scale microchannel devices is to form the microchannels in the shims by different methods such as etching, stamping etc. These techniques are known in the art. For example, shims may be stacked together and joined by different methods such as chemical bonding, brazing etc. After joining, the device may or may not require machining.

In some embodiments, the inventive apparatus (or method) includes a catalyst material. The catalyst may define at least a portion of at least one wall of a bulk flow path. In some preferred embodiments, the surface of the catalyst defines at least one wall of a bulk flow path through which passes a fluid stream. During a hetereogeneous catalysis process, a reactant composition can flow through a microchannel, past and in contact with the catalyst.

In preferred embodiments, the width of each connecting microchannel is substantially constant along its length and each channel in a set of connecting channels have substantially constant widths; “substantially constant” meaning that flow is essentially unaffected by any variations in width. For these examples the width of the microchannel is maintained as substantially constant. Where “substantially constant” is defined within the tolerances of the fabrication steps. It is preferred to maintain the width of the microchannel constant because this width is an important parameter in the mechanical design of a device in that the combination of microchannel width with associated support ribs on either side of the microchannel width and the thickness of the material separating adjacent lamina or microchannels that may be operating at different temperatures and pressures, and finally the selected material and corresponding material strength define the mechanical integrity or allowable temperature and operating pressure of a device.

In some preferred embodiments, connecting microchannels do not have surface features. In some embodiments, microchannel devices do not have gates, grates, flow straighteners, or orifices to regulate flow into connecting channels. In some preferred embodiments, flow is distributed via submanifolds to multiple connecting channels.

Microchannels (with or without surface features) can be coated with catalyst or other material such as sorbent. Catalysts can be applied onto the interior of a microchannel using techniques that are known in the art such as wash coating. Techniques such as CVD or electroless plating may also be utilized. In some embodiments, impregnation with aqueous salts is preferred. Pt, Rh, and/or Pd are preferred in some embodiments. Typically this is followed by heat treatment and activation steps as are known in the art. Other coatings may include sol or slurry based solutions that contain a catalyst precursor and/or support. Coatings could also include reactive methods of application to the wall such as electroless plating or other surface fluid reactions.

For microchannel devices with M2M manifolds within the stacked shim architecture, the M2M manifolds add to the overall volume of the device and so it is desirable to maximize the capacity of the manifold. In preferred embodiments of the invention, an M2M distributes at least 0.1 kg/m³/s, preferably 1 kg/m³/s or more, more preferably at least 10 kg /M³s, and in some preferred embodiments distributes 30 to 5000 kg/m³/s, and in some embodiments 30 to 1000 kg/m³/s.

The invention includes processes of conducting chemical reactions and other unit operations in the apparatus described herein. The invention also includes prebonded assemblies and laminated devices of the described structure and/or formed by the methods described herein. Laminated devices can be distinguished from nonlaminated devices by optical and electron microscopy or other known techniques. The invention also includes methods of conducting chemical processes in the devices described herein and the methods include the steps of flowing a fluid through a manifold and conducting a unit operation in the connecting channels (if the manifold is a header, a fluid passes through the manifold before passing into the connecting channels; if the manifold is a footer then fluid flows in after passing through the connecting channels). In some preferred embodiments, the invention includes non-reactive unit operations, including heat exchangers, mixers, chemical separators, solid formation processes within the connecting channels, phase change unit operations such as condensation and evaporation, and the like; such processes are generally termed chemical processes, which in its broadest meaning (in this application) includes heat exchange, but in preferred embodiments is not solely heat exchange but includes a unit operation other than heat exchange and/or mixing.

The invention also includes processes of conducting one or more unit operations in any of the designs or methods of the invention. Suitable operating conditions for conducting a unit operation can be identified through routine experimentation. Reactions of the present invention include: acetylation, addition reactions, alkylation, dealkylation, hydrodealkylation, reductive alkylation, amination, ammoxidation aromatization, arylation, autothermal reforming, carbonylation, decarbonylation, reductive carbonylation, carboxylation, reductive carboxylation, reductive coupling, condensation, cracking, hydrocracking, cyclization, cyclooligomerization, dehalogenation, dehydrogenation, oxydehydrogenation, dimerization, epoxidation, esterification, exchange, Fischer-Tropsch, halogenation, hydrohalogenation, homologation, hydration, dehydration, hydrogenation, dehydrogenation, hydrocarboxylation, hydroformylation, hydrogenolysis, hydrometallation, hydrosilation, hydrolysis, hydrotreating (including hydrodesulferization HDS/HDN), isomerization, methylation, demethylation, metathesis, nitration, oxidation, partial oxidation, polymerization, reduction, reformation, reverse water gas shift, Sabatier, sulfonation, telomerization, transesterification, trimerization, and water gas shift. For each of the reactions listed above, there are catalysts and conditions known to those skilled in the art; and the present invention includes apparatus and methods utilizing these catalysts. For example, the invention includes methods of amination through an amination catalyst and apparatus containing an amination catalyst. The invention can be thusly described for each of the reactions listed above, either individually (e.g., hydrogenolysis), or in groups (e.g., hydrohalogenation, hydrometallation and hydrosilation with hydrohalogenation, hydrometallation and hydrosilation catalyst, respectively). Suitable process conditions for each reaction, utilizing apparatus of the present invention and catalysts that can be identified through knowledge of the prior art and/or routine experimentation. To cite one example, the invention provides a Fischer-Tropsch reaction using a device (specifically, a reactor) having one or more of the design features described herein.

Pressure drop through a set of connecting microchannels is preferably less than 500 psi, more preferably less than 50 psi and in some embodiments is in the range of 0.1 to 20 psi. In some embodiments, wherein the manifold is a header, the pressure drop in the manifold, as measured in psi between the header inlet and the connecting channel inlet (corresponding to a header outlet) having the lowest pressure, is less than (more preferably less than 80% of, more preferably less than half (50%) of, and in some embodiments less than 20% of) the pressure drop through the plural connecting channels (measured as an average pressure drop over the plural connecting channels).

In some preferred embodiments, the manifold volume is less than 80%, or less than 50% (half) in some embodiments 40% or less, and in some embodiments less than 20% of the volume of the plural connecting channels. In some embodiments, the manifold volume is 10% to 80% of the volume of the plural connecting channels. Preferably, the combined volume of all manifolds in a laminated device is 50% or less, in some embodiments 40% or less, of the combined volume of all connecting channels in a device; in some embodiments, 10% to 40%.

Quality Index factor “Q₁” is a measure of how effective a manifold is in distributing flow. It is the ratio of the difference between the maximum and minimum rate of connecting channel flow divided by the maximum rate. For systems of connecting channels with constant channel dimensions it is often desired to achieve equal mass flow rate per channel. The equation for this case is shown below, and is defined as Q₁.

$${Q}_{1} = \frac{{m}_{max} - {m}_{min}}{{m}_{max}} \times 100 \%$$

where
mₘₐₓ[kg/sec]=maximum connecting channel mass flow rate
mₘᵢₙ[kg/sec]=minimum connecting channel mass flow rate
For cases when there are varying connecting channel dimensions it is often desired that the residence time, contact time, velocity or mass flux rate have minimal variation from channel to channel such that the required duty of the unit operation is attained. For those cases we define a quality index factor Q₂:

$${Q}_{2} = \frac{{G}_{max} - {G}_{min}}{{G}_{max}} \times 100 \% ,$$

where G is the mass flux rate. For cases when all the connecting channels have the same cross sectional area (as in some embodiments of the invention), the equation for Q₂ simplifies to Q₁. The quality index factor gives the range of connecting channel flow rates, with 0% being perfect distribution, 100% showing stagnation (no flow) in at least one channel, and values of over 100% indicating backflow (flow in reverse of the desired flow direction) in at least one channel. Q₁ and Q₂ are defined based on the channels that comprise 95% of the net flow through the connecting channels, the lowest flow channels are not counted if the flow through those channels is not needed to account for 95% of the net flow through the connecting channels. In methods of the present invention, the Quality factor is preferably 10% or less, more preferably 5%, and still more preferably 1% or less; and in some embodiments is in the range of 0.5% to 5%.

Q factor can also be used as a metric to characterize apparatus containing connecting channels. In preferred embodiments, the inventive apparatus can be characterized by a Q factor (Q₁) of 10% or less, more preferably 5% or less, or 2% or less, or in some embodiments, in the range of 0.5% to 5%). To determine the Q factor property of a device, air is flowed through the device at 20° C. and Mo=0.5. The distribution through connecting channels can be measured directly or from computational fluid dynamic (CFD) modeling.

Heat exchangers made using a partial etch or material removal from a laminate are particularly advantageous for this application. Channel gaps are preferably in the range of 0.5 to 1.5 mm and thus a minimum number of laminates are required during manufacturing. The depth of the channel is removed from a laminate leaving a wall that intervenes between flow channels, and preferably ribs that support the walls for the differential pressure at temperature and preferably create a high aspect ratio microchannel (width to gap ratio>2). In some embodiments, flow straighteners and modifiers are disposed in an M2M section.

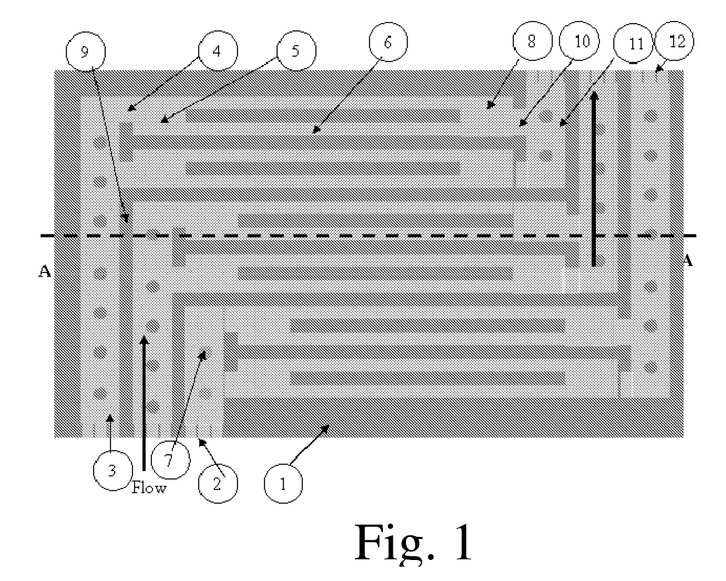

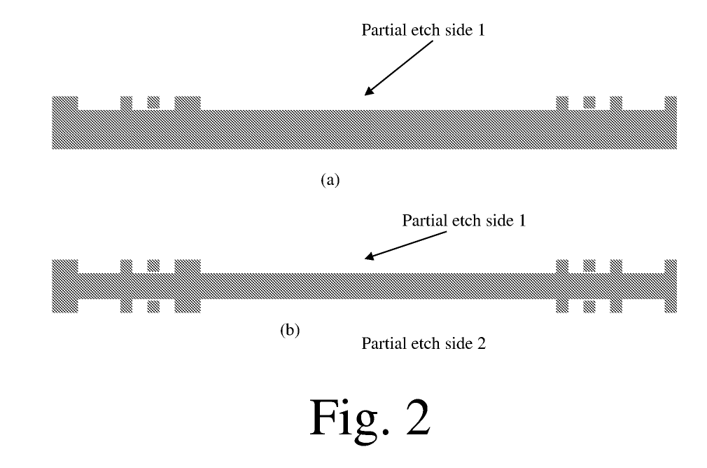

FIG. 1 shows a schematic of general concept of manifold, connecting channels and the connections in between on a shim. The shim can be made by partial etching out of any material (metal, polymer etc). In one embodiment, the shim was etched only on one side. In another embodiment, the shim is etched from both sides as shown by cross-sectional view of section A-A in FIG. 2. It should be understood that methods other than a chemical etching may create similar features. In the embodiment when the shim is etched on both sides, the depth of etching on one side of the shim may be different or similar to the depth of the etching on the other side.

A fluid enters the shim through 2 which are multiple small cross-sectional openings. The flow then enters 3 which is referred as inlet sub-manifold. The inlet sub-manifolds are separated from each other through ribs 9.

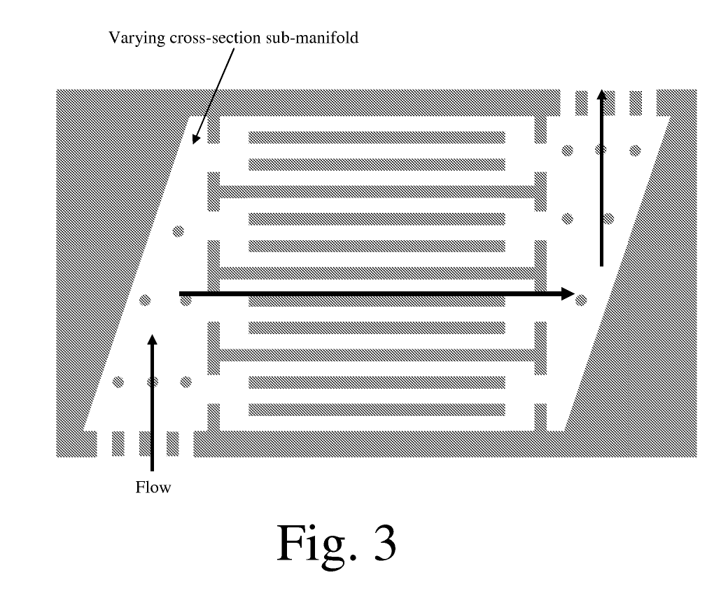

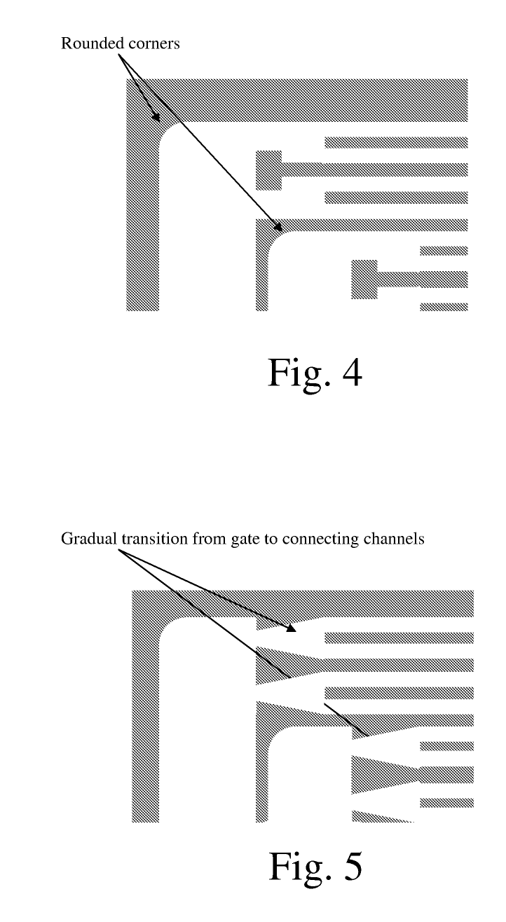

In some embodiments an inlet sub-manifold is rectangular in cross-section as shown in FIG. 1. In another embodiment, the inlet sub-manifold has varying cross-section as shown in FIG. 3. The variation in the cross-section of the inlet sub-manifold can be continuous (as shown in FIG. 3) or in steps. An inlet submanifold can increase or decrease in cross sectional area in the direction of length toward the connecting channels. In one embodiment the inlet sub-manifold has sharp corners. In another embodiment the sub-manifold has rounded corners as shown in FIG. 4.

For a given space for inlet sub-manifolds in a shim, the number of inlet sub-manifolds in a shim can be increased by reducing the rib between the sub-manifolds.

Within each inlet sub-manifold, pressure support features, 7, can be present which may or may not be required. The pressure support features can be in any shape or size however the height of these features is same as the depth of the etching. These features support the differential pressure between the streams in the inlet sub-manifold section. Also the features act as obstructions and may increase pressure drop. The shape, size and number of pressure support features should be determined from the overall pressure drop requirements and stress requirements.

The flow from inlet sub-manifolds can enter inlet gates 4 followed by inlet flow straightener 5. In one embodiment, one inlet sub-manifold has 2 inlet gates. In another embodiment, one inlet sub-manifold has the number of inlet gates equal to number of connecting channels, 6 (not shown).The size of the inlet gates is preferably controlled to provide highly uniform flow distribution in the connecting channels.

The inlet flow straightner removes any directional component of flow perpendicular to connecting channels and hence may or may not be required. In one embodiment the transition of the flow from the inlet gates to the connecting channels is abrupt through the inlet flow straightner as shown in FIG. 1. In another embodiment the transition of the flow from the inlet gates to the connecting channels is gradual as shown in FIG. 5 with preferably increasing cross sectional area from the submanifold to the connecting channels. As mentioned, the gate volume is counted as part of the manifold volume. The corners of inlet gates and inlet flow straightners can be sharp or rounded.

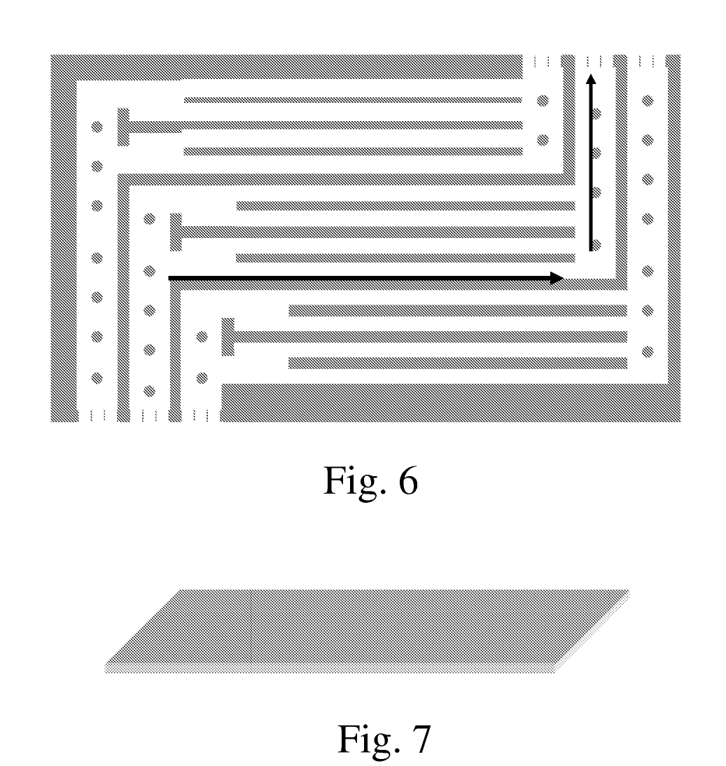

The flow then enters the connecting microchannels. The number of connecting channels may be varied from submanifold to submanifold or may be similar across the width of the shim. The connecting channels are separated from each other by ribs that do not allow the flow to communicate in the process channels. In an alternate embodiment, the ribs may be discontinuous and permit some fluid communication between parallel microchannels. In this embodiment, the fluid communication may permit a flow redistribution and improved or a reduced quality index. The flow will then exit the device through exit flow straightner 8, exit gate 10, exit sub-manifold 11 and exit openings 12. In the illustrated embodiment, exit flow straightners, exit gates and exit sub-manifolds have the same characteristics as inlet flow straightner, inlet gates and inlet sub-manifolds respectively. The connecting channels can be directly connected to an exit sub-manifold as shown in FIG. 6. In another embodiment, the inlet sub-manifolds are directly connected to the channels while exit flow straightner, exit gates, exit sub-manifolds are used at the exit of the device.

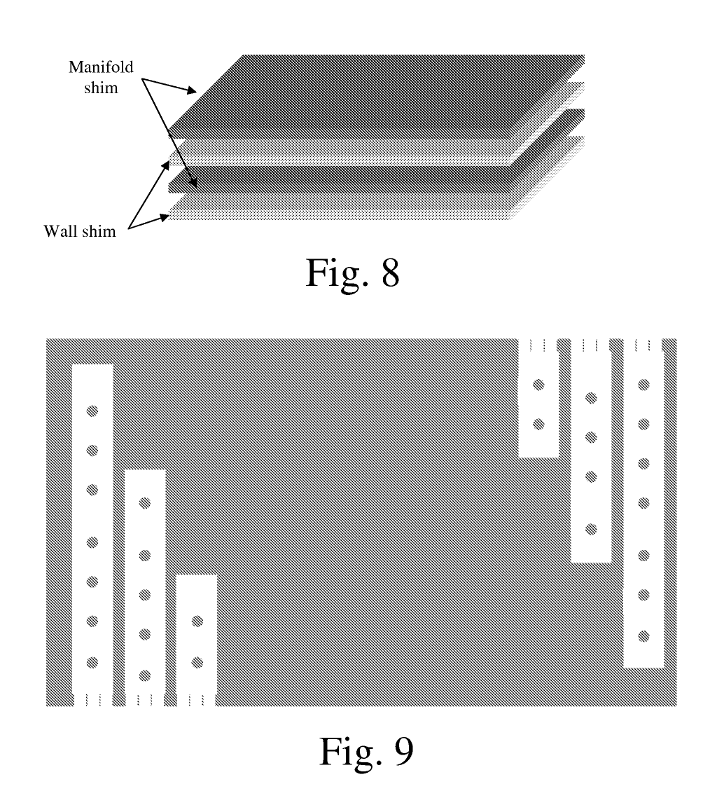

FIG. 7 shows a wall shim. FIG. 8 shows the assembly of a device by stacking manifold and wall shims to develop a device stack. The manifold shims and wall shims are repeated in a similar fashion in the stack to create the device stack. In one embodiment at least one manifold shim is different from the other manifold shims in the stack. In another embodiment, all the manifold shims are different in design from other manifold shims.

In one embodiment, the some of the wall shims in the stack assembly have sub-manifold similar to manifold shim such that after stacking it with manifold shims, the sub-manifold in the manifold shims and in the wall shims are aligned. An example of such a wall shim embodiment is shown in FIG. 9. The flow enters the sub-manifold sections of manifold shim and the wall shim and then splits into manifold shims to flow in the gates and connecting channels. At the exit sub-manifold, the flow in two sub-manifold shims recombines and leaves the device.

In one embodiment, the flow distribution features and micromanifold for one fluid stream including gates, grates, posts, flow straighteners and the like may be disposed at positions along the length of the device that do not correspond with the flow distribution features and micromanifold for at least one second stream in a multistream heat exchanger, or other unit operation. For example, fluid flow paths in adjacent layers may have flow distribution features and manifolds that do not correspond between layers.

In some preferred embodiments, three or more fluid streams are used in the inventive device to transfer heat, mix fluids, conduct a reaction, and or conduct a separation. It may be preferential for similar fluid streams to be adjacent to each other in the process channels such that the micromanifold section may be preferably made with a channel gap (“gap” is measured in the stacking direction) greater than the channel gap in the connecting channel.

In some preferred embodiments, the number of submanifolds is set to reduce the total flowrate in any submanifold such that laminar flow is maintained. Laminar-only flow in the submanifold will result in a lower pressure drop per unit length than a transition or turbulent flow.

The use of disrupted flow for chemical reactions, separation, or mixing is particularly advantageous in a portion of the connecting channels that is at least 5% of the connecting channel length. The use of disrupted flow as applied to mass exchange unit operations (reaction, separation and/or mixing) allow for enhanced performance with process channel gaps in the preferred range of 0.5 mm to 1.5 mm which concurrently enable a more compact M2M than mass exchange applications with smaller microchannels operating in laminar flow in the connecting channels. As an example for a heterogeneous reaction, the use of disrupted flow to bring reactants to the catalyst on the wall versus laminar diffusion to bring reactants to the catalyst overcomes mass transfer limitations. The effective performance of a catalyst may be 2 or more or 5, or 10, or 100 or 1000 times or more effective than laminar only flow. The more effective mass transfer performance for the catalyst enables a smaller volume for the connecting channels while also permitting channel gaps in the M2M to remain in the preferred region of 0.5 to 1.5 mm and thus minimizes the M2M volume. Chemical separation examples also include absorption, adsorption, distillation, membrane and the like. Chemical separation, mixing, or chemical reactions are particularly optimized for total volume minimization of M2M plus connecting channel volume if at least a portion of the connecting channel is in disrupted flow.

### EXAMPLE

### Calculated Comparison of Two Heat Exchanger Designs

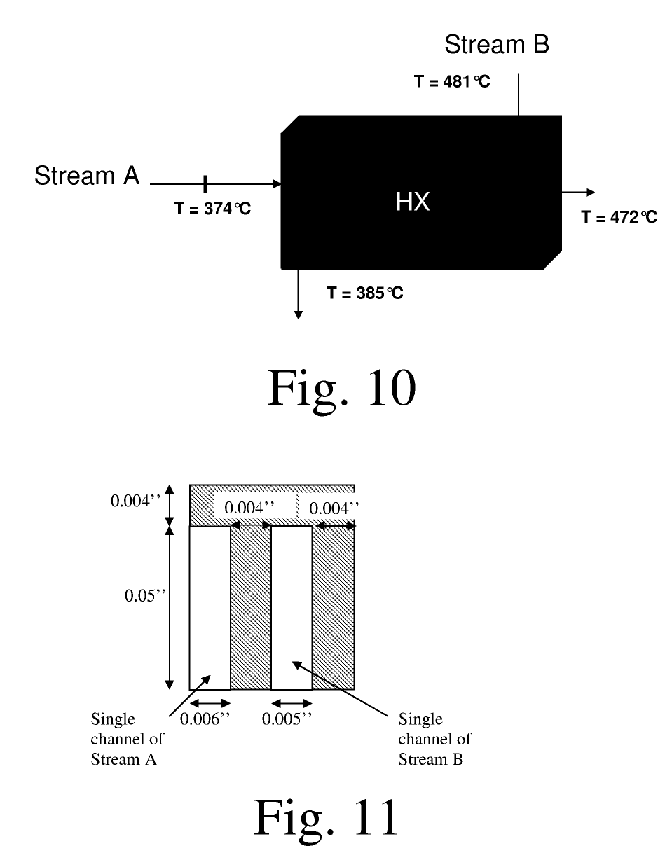

Two heat exchanger designs were compared: One with large microchannels and other with smaller microchannels. The heat exchanger was a two stream counter-current heat exchanger as shown in the FIG. 10. Table 1 lists the inlet conditions and outlet requirements for the two streams.

TABLE 1
Inlet conditions and outlet requirements for heat exchanger

| Condition | Stream A | | Stream B | |
|---|---|---|---|---|
| Mass flow rate (kg/hr) | 202604 | kg/hr | 202604 | kg/hr |
| Inlet temperature (° C.) | 374° | C. | 481° | C. |
| Desired outlet temperature (° C.) | 472° | C. | 385° | C. |
| Outlet pressure (psig) | 349.8 | psig | 323.3 | psig |
| Allowable pressure drop (psi) | 4.0 | psi | 3.0 | psi |

The composition of Stream A and Stream B are summarized below in Table 2.

TABLE 2
Molar composition of Stream A and Stream B

Molar Composition (%)

| Component | Stream A | Stream B |
|---|---|---|
| Water | 57.01% | 69.20% |
| Nitrogen | 0.78% | 0.84% |
| Hydrogen | 10.29% | 0.76% |
| Carbon-monoxide | 0.11% | 0.02% |
| Carbon-dioxide | 3.97% | 0.31% |
| Methane | 27.83% | 24.97% |
| Ethane | 0.00% | 2.03% |
| Propane | 0.00% | 0.82% |
| n-butane | 0.00% | 0.47% |
| n-pentane | 0.00% | 0.06% |
| Methanol | 0.00% | 0.51% |

The thermo-physical properties (specific heat, thermal conductivity, viscosity) of Stream A and Stream B were calculated using ChemCAD V5.5x. The density of the Stream A and Stream B were calculated as ideal gas law.
Design 1: Small Microchannel Design
Design of Core Section

The arrangement of the two streams in a repeating unit of the core section is shown below:
---Stream A---Stream B---Stream A---Stream B---Stream A---Stream B---
   The dimensions of a single repeating unit are shown in the FIG. 11. The flow direction is perpendicular to the plane of the figure. The connecting channel opening for Stream A was 0.05″×0.006″ while for Stream B was 0.05″×0.005″. The thickness of wall was 0.004″ everywhere in the repeating unit. The repeating unit is expanded in direction perpendicular to the flow to obtain the core section.

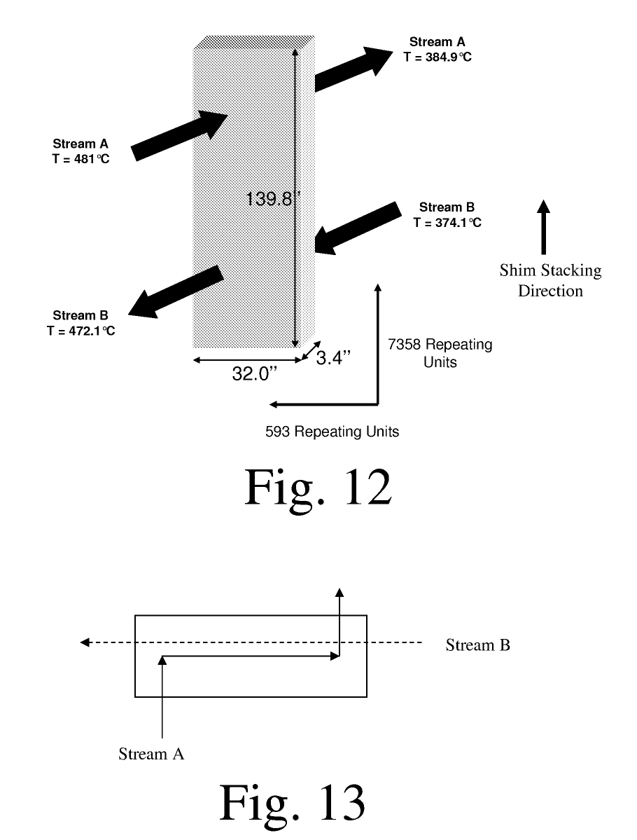

The length of heat exchanger core required for heat transfer was 3.4″. The number of repeating units in shim stacking direction was 7358 while the number of repeating units in a shim was 593. The predicted outlet temperature of streams is also shown in the FIG. 12. The average Reynolds number of the hot stream was 722 while the average Reynolds number for cold stream was 762 approximately. The predicted pressure drop for Stream A and Stream B are shown in Table 3.

TABLE 3
Predicted pressure drop for Design 1 - Core Section
Predicted Pressure Drop (psi)

| Stream A | Stream B |
|---|---|
| 2.0 psi | 2.8 psi |

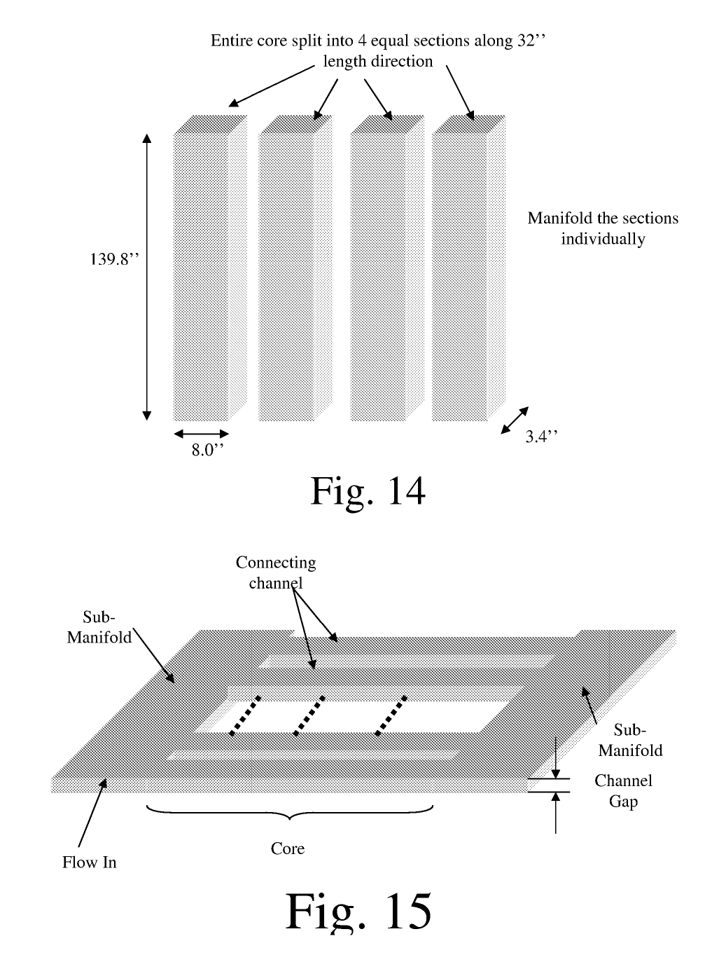

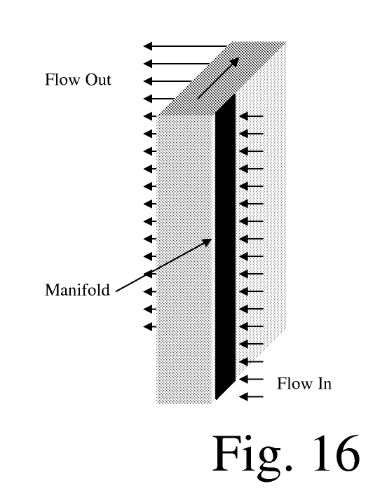

Total heat transferred in the core section was 13.7 MW.
Design of Manifold Section for Distributing Flow in Microchannels
Assumptions made in design of Manifold section are listed below:
1. There is no heat transfer in manifold section
2. Stream A has a Z-manifold design while Stream B went straight through as shown in FIG. 13. So the internal manifold was designed only for Stream A.
3. The core was divided into 4 sections along 32.0″ dimension (593 repeating units) and then the internal manifold was designed for each section as shown in FIG. 14.
   The gap available for flow in manifold section is same as the main channel gap as shown in FIG. 15. FIG. 16 shows the sketch of flow entrance and exit into one of the four core section of the device.

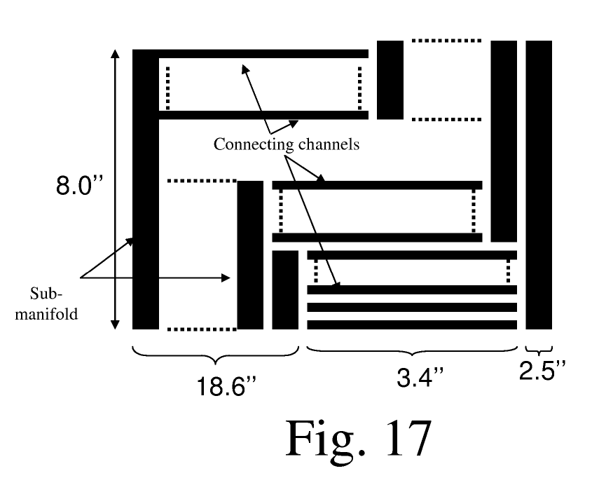

The flow enters the sub-manifold and distributes the flow in connecting channels in the heat exchanger core section. To distribute the flow in the one of the four core sections, more than one sub-manifolds are required. The picture of manifold design illustrating the dimensional requirements for uniform distribution of Stream A in one of the four core sections is shown in the FIG. 17.

The geometry shown in FIG. 17 can be etched on a shim and will be the footprint of a single core section. If a metal allowance of 0.25″ is given on the shim at the perimeter and 0.25″ for the end plate thickness then the overall size of a single heat exchanger core with manifold will be: 25.0−×8.5″×140.3″. The total volume of the heat exchanger (four cores) will be 119,260 in³. The volume of the connecting channels for A was only 14% of the total volume inclusive of the manifold volume.

Design 2: Large Microchannel Design

The same design strategy was used for designing the heat exchanger with larger microchannels. The repeated unit in the core section is shown below:

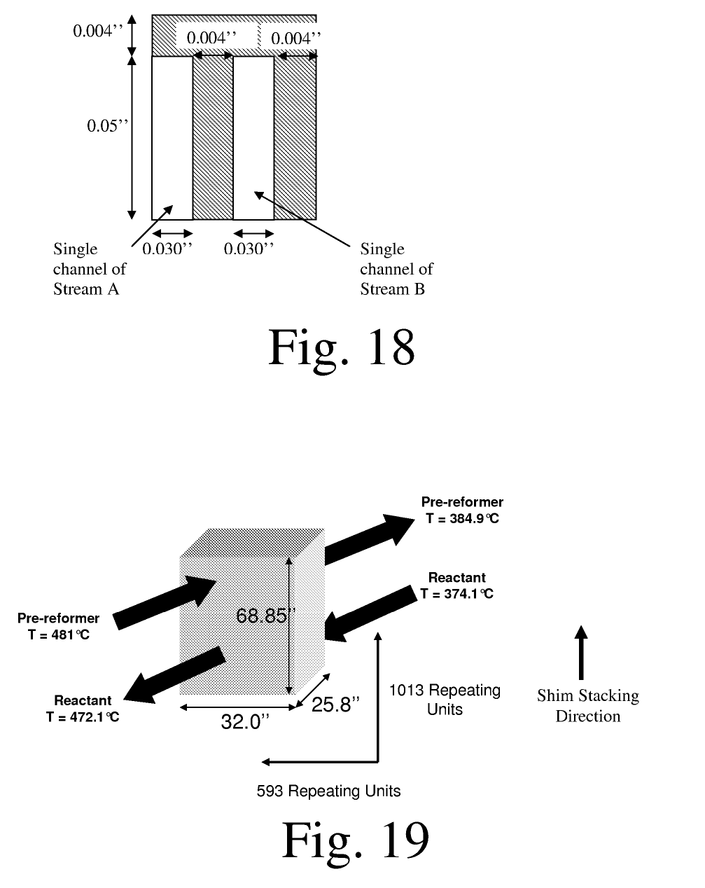

---Stream A---Stream B---Stream A---Stream B---Stream A---Stream B---
   The dimensions of a single repeating unit are shown in the FIG. 18. The flow direction is perpendicular to the plane of the figure. The channel dimension for Stream A was 0.05″×0.03″ while for Stream B was 0.05″×0.03″. The thickness of wall was 0.004″ everywhere in the repeating unit. The repeating unit is expanded in direction perpendicular to the flow to obtain the core section.

The overall size of the core estimated is shown in FIG. 19. The number of repeating units in shim stacking direction was 1013 while the number of repeating units in a shim was 593. length of heat exchanger core required was 25.8″. The predicted outlet temperature of streams is also shown in the FIG. 19. The average Reynolds number of the hot stream was 3670 while the average Reynolds number of cold stream was 3810 approximately. The use of transition to low turbulent flow in the microchannel creates higher heat transfer coefficients such that a larger microchannel gap of 0.03″ is acceptable relative to the heat transfer coefficient for a laminar flow stream in a 0.03″ channel gap. The predicted pressure drop for Stream A and Stream B are shown in Table 4.

TABLE 3
Predicted pressure drop for Design 2 - Core Section
Predicted Pressure Drop (psi)

| Stream A | Stream B |
|---|---|
| 2.5 psi | 2.9 psi |

Total heat transferred in the core section was 13.7 MW.

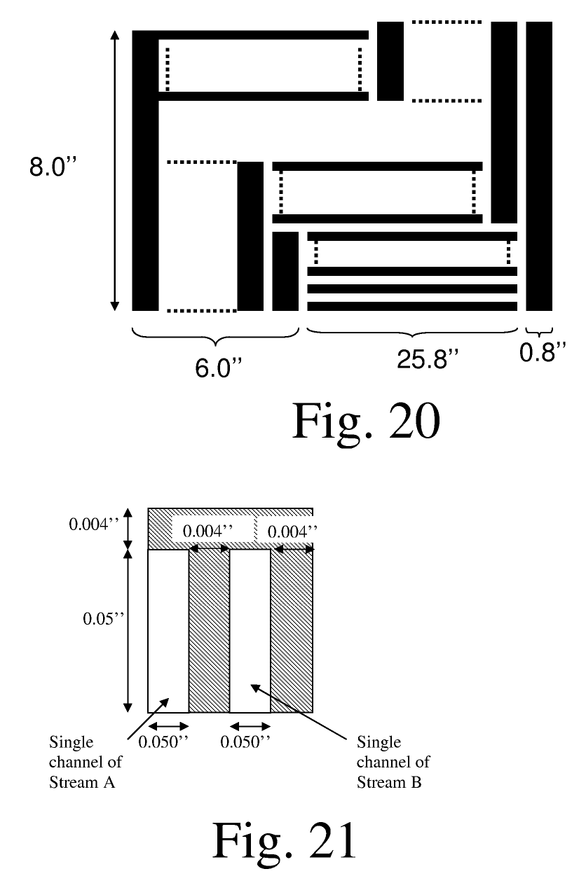

The design for distributing stream A in one of the four cores is shown in the FIG. 20.

If a metal rim of 0.25″ is given on the shim at the perimeter then the overall size of a single heat exchanger core with manifold will be: 33.1″×8.5″×69.4″. The total volume of the heat exchanger (four cores) will be 78,100 in³. The volume of the connecting channel was 79% of the total volume inclusive of the manifold volume.

Design 3: Large Microchannel Design—2

The same design strategy was used for designing the heat exchanger with even larger microchannels. The repeated unit in the core section is shown below:

---Stream A---Stream B---Stream A---Stream B---Stream A---Stream B---
   The dimensions of a single repeating unit are shown in the FIG. 21. The flow direction is perpendicular to the plane of the figure. The channel dimension for Stream A was 0.05″×0.05″ while for Stream B was 0.05″×0.05″. The thickness of wall was 0.004″ everywhere in the repeating unit. The repeating unit is expanded in direction perpendicular to the flow to obtain the core section.

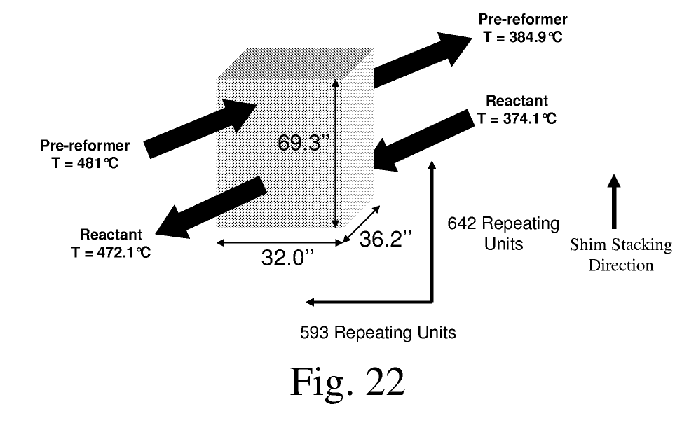

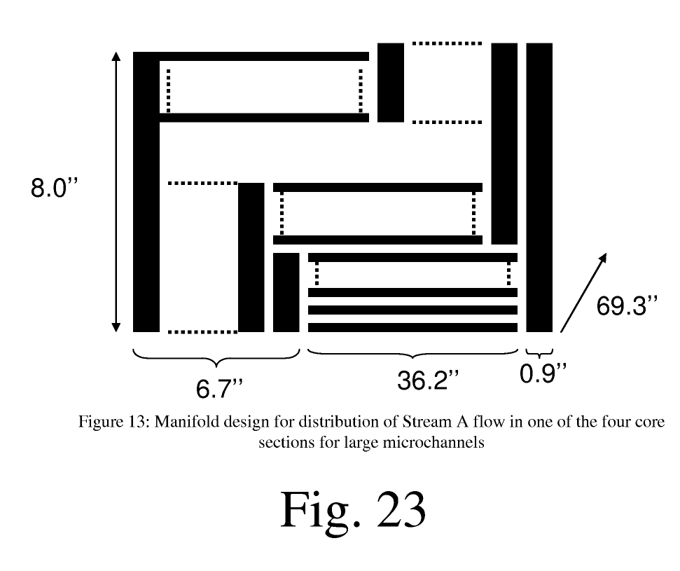

The overall size of the core estimated is shown in FIG. 22. The number of repeating units in shim stacking direction was 641 while the number of repeating units in a shim was 593. Length of heat exchanger core required was 36.2″. The predicted outlet temperature of streams is also shown in the FIG. 21. The average Reynolds number of the hot stream was 4650 while the average Reynolds number of cold stream was 4800 approximately. The predicted pressure drop for Stream A and Stream B are shown in Table 4.

TABLE 3
Predicted pressure drop for Design 2 - Core Section
Predicted Pressure Drop (psi)

| Stream A | Stream B |
|---|---|
| 2.5 psi | 2.9 psi |

Total heat transferred in the core section was 13.7 MW.

The design for distributing stream A in one of the four cores is shown in the FIG. 23.

If a metal rim of 0.25″ is given on the shim at the perimeter then the overall size of a single heat exchanger core with manifold will be: 44.3″×8.5″×69.8″. The total volume of the heat exchanger (four cores) will be 105,133 in³. The volume of the connecting channel was 82% of the total volume inclusive of the manifold volume.

Table 5 compares the size and performance of designs with small microchannels and large microchannels.

| | Design 1: Small | | Design 2: Large | | Design 3: Large | |
|---|---|---|---|---|---|---|
| | Microchannels | | microchannels | | microchannels | |
| Total Heat | 13.7 | MW | 13.7 | MW | 13.7 | MW |
| Duty (MW) | | | | | | |
| Channel | 0.006″ | | 0.03″ | | 0.05″ | |
| gap (in) | | | | | | |
| Pressure | | | | | | |
| drop (psi) | | | | | | |
| Stream A | 4.0 | psi | 4.0 | psi | 3.4 | psi |
| Stream B | 2.8 | psi | 2.5 | psi | 2.5 | psi |
| Quality | <5% | (1.3%) | <5% | (4%) | <5% | (4%) |
| Factor (%) | | | | | | |
| Overall | 119,260 | in³ | 78,100 | in³ | 105,133 | in³ |
| Size (in³) | | | | | | |

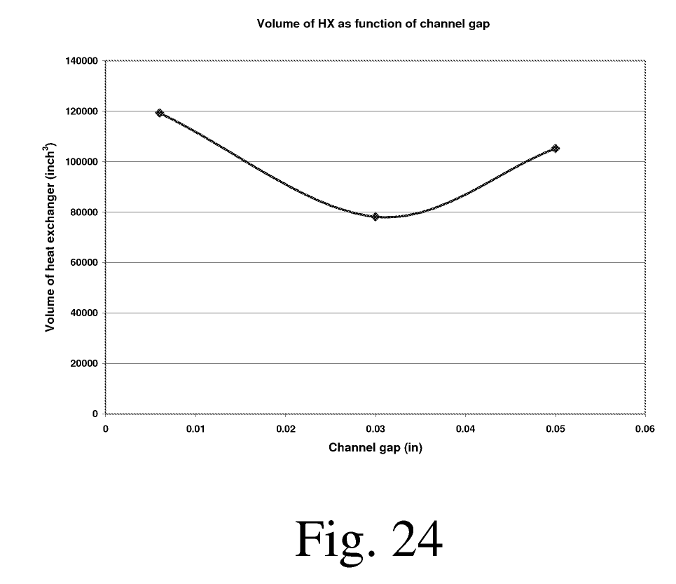

In summary, small channel gap as taught by literature does not always lead to best design. Microchannels in the range of 0.5 mm to 1.5 mm may be large enough to have transition or turbulent flow regime which provides good convective heat transfer properties and the larger gaps provide enough space to manifold the flow in a relatively small volume. For the above example, variation of overall device volume as a function of channel gap is illustrated in FIG. 24.

1. A method of conducting a unit operation in an integrated microchannel apparatus, comprising:
passing a fluid in an apparatus;
wherein the apparatus comprises a manifold connected to plural connecting microchannels;
wherein the manifold's volume is less than the volume of the plural connecting microchannels;
wherein the manifold's length is at least 15 cm or wherein there are at least 100 connecting channels connected to the manifold;
controlling conditions such that the fluid is in disrupted flow through at least a portion of the connecting microchannels; and
conducting a unit operation on the fluid in the connecting microchannels.

2. The method of claim 1 wherein the device comprises at least two manifolds, a first manifold and a second manifold, wherein the first manifold is connected to a first set of plural connecting microchannels and the second manifold is connected to a second set of plural connecting microchannels.

3. The method of claim 2 wherein a first fluid flows through the first manifold and flows in disrupted flow substantially through the first set of connecting microchannels and wherein a second fluid flows through the second manifold and flows in non-disrupted flow substantially through the second set of connecting microchannels.

4. The method of claim 1 wherein the manifold is a header and wherein the header has an inlet, and wherein fluid passes through the header inlet at a Reynold's number greater than 2200.

5. The method of claim 1 wherein the integrated microchannel apparatus has a heat duty greater than 0.01 MW.

6. The method of claim 1 wherein pressure drop through the manifold is less than or equal to the average pressure drop through connecting channels.

7. The method of claim 1 wherein the manifold is a header and wherein the pressure drop in the manifold, that is between the header inlet and the connecting channel inlet (corresponding to a header outlet) having the lowest pressure, is less than 50% of the pressure drop through the plural connecting channels (measured as an average pressure drop).

8. The method of claim 1 wherein the manifold is a header and wherein the pressure drop in the manifold, that is between the header inlet and the connecting channel inlet (corresponding to a header outlet) having the lowest pressure, is less than 25% of the pressure drop through the plural connecting channels (measured as an average pressure drop).

9. The method of claim 1 wherein the manifold volume is less than 50% of the volume of the plural connecting channels.

10. The method of claim 1 wherein the manifold volume is less than 25% of the volume of the plural connecting channels.

11. The method of claim 1 wherein the integrated microchannel apparatus has a heat duty greater than 0.1 MW.

12. The method of claim 1 wherein the integrated microchannel apparatus has a heat duty greater than 1 MW.

13. The method of claim 1 wherein there are no orifices controlling flow between the manifold and the connecting channels; wherein an orifice's cross-sectional area is defined as less than 20% of the average cross-sectional area of the connecting channels.

14. The method of claim 1 wherein the manifold comprises two sections.

15. The method of claim 14 wherein two sections comprise a first and a second section, and wherein the first section is an open manifold and the second section comprises a submanifold, gate, or grate.

16. Microchannel apparatus, comprising:
a manifold connected to plural connecting microchannels;
wherein the manifold's volume is less than the volume of the plural connecting microchannels;
wherein the manifold's length is at least 15 cm or wherein there are at least 100 connecting channels connected to the manifold.

17. The apparatus of claim 16 comprising at least 10 layers of heat exchange microchannel arrays interfaced with at least 10 layers of reaction microchannels, wherein the reaction microchannels comprise a catalyst wall coating.

18. The apparatus of claim 16 wherein each layer of heat exchange microchannel arrays comprises a manifold and an array of heat exchange connecting microchannels connected to the manifold.

19. The apparatus of claim 18 wherein the manifold in each layer is substantially limited to that layer and does not extend over plural layers of heat exchange microchannel arrays.

20. The apparatus of claim 19 wherein a manifold extends over plural layers of heat exchange microchannel arrays such that plural arrays of heat exchange connecting microchannels in plural layers connect to the manifold.

21. A microchannel system comprising a device and a fluid, comprising:
a manifold connected to plural connecting microchannels;
wherein the manifold's volume is less than the volume of the plural connecting microchannels;
wherein the manifold's length is at least 15 cm or wherein there are at least 100 connecting channels connected to the manifold; and
a fluid passing through the connecting microchannels in disrupted flow for at least a portion of the length.

22. A method of conducting a unit operation in an integrated microchannel apparatus, comprising:
passing a fluid in an apparatus;
wherein the apparatus comprises a manifold connected to plural connecting microchannels;
wherein the manifold's volume is less than the volume of the plural connecting microchannels;
controlling conditions such that the fluid is in disrupted flow substantially through at least some the plural connecting microchannels and controlling conditions such that the fluid is in non-disrupted flow substantially through at least some other of the plural connecting microchannels; and
conducting a unit operation on the fluid in the connecting microchannels that are in disrupted flow and conducting a unit operation on the fluid in the connecting microchannels that are in non-disrupted flow.

23. The method of claim 1 wherein flow through the plural connecting channels is transitional or turbulent flow.

24. The method of claim 23 wherein the plural connecting channels have smooth walls.

25. The method of claim 23 wherein the plural connecting channels do not have surface features.

26-37. (canceled)
